(19) United States
(12) Patent Application Publication
(10) Pub. No.: US 2024/0299326 A1
(43) Pub. Date: Sep. 12, 2024

(54) METHODS AND COMPOSITIONS FOR TREATING CANCER

(71) Applicant: Onconova Therapeutics, Inc., Newtown, PA (US)

(72) Inventors: Steven Fruchtman, New York, NY (US); Matthew Parris, Plainsboro, NJ (US)

(21) Appl. No.: 18/437,793

(22) Filed: Feb. 9, 2024

Related U.S. Application Data
(63) Continuation of application No. PCT/US22/40092, filed on Aug. 11, 2022.
(60) Provisional application No. 63/232,409, filed on Aug. 12, 2021.

(51) Int. Cl. A61K 31/198; A61K 9/00; A61K 9/08; A61K 9/48; A61P 17/00; A61P 35/00

(52) U.S. Cl. CPC A61K 31/198; A61K 9/0019; A61K 9/0053; A61K 9/08; A61K 9/48; A61P 17/00; A61P 35/00

(57) ABSTRACT

Disclosed herein is a method of treating a squamous cell carcinoma (SCC), a SCC associated with a disease, for example, recessive dystrophic epidermolysis bullosa (RDEB). The method of the disclosure describes administering a therapeutically-effective amount of (E)-2-(5-((2,4,6-trimethoxystyrylsulfonyl)methyl)-2-methoxyphenylamino)acetic acid, or a pharmaceutically-acceptable salt or zwitterion thereof, and derivatives thereof.

### CROSS-REFERENCE

This application is a continuation of International Application No. PCT/US22/40092 filed Aug. 11, 2022, which claims the benefit of U.S. Provisional Application No. 63/232,409 filed Aug. 12, 2021, each of which is entirely incorporated herein by reference.

### BACKGROUND

Some lung adenocarcinomas have a KRAS mutation as a predominant genetic driver. Tumors carrying a mutation in KRAS can have a worse prognosis than KRAS wild-type tumors have. Therapies that can target the KRAS pathway could be beneficial for patients with cancers having KRAS mutations.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

Disclosed herein is a method of treating a condition in a subject in need thereof, the method comprising administering to the subject a therapeutically-effective amount of a compound of the formula:

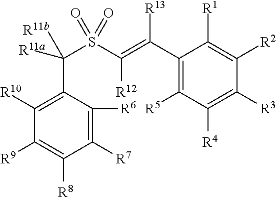

or a pharmaceutically-acceptable salt or zwitterion thereof,
wherein:
each R¹, R², R³, R⁴, R⁵, R⁶, R⁷, R⁸, R⁹, R¹⁰, R^{11a}, R^{11b}, R¹², and R¹³ is independently alkyl, alkenyl, alkynyl, alkoxy, aryl, heteroaryl, heterocyclyl, C(O)R^{x}, C(O)OR^{x}, C(O)NR^{x}R^{y}, OR^{x}, SR^{x}, NRR^{y}, NR^{x}C(O)R^{y}, OC(O)R^{x}, or SiR^{x}R^{y}R^{z}, each of which is independently substituted or unsubstituted; or hydrogen or halogen; and
each R^{x}, R^{y}, and R^{z} is independently alkyl, alkenyl, alkynyl, aryl, heteroaryl, or heterocyclyl, each of which is independently substituted or unsubstituted; or hydrogen or halogen,
   wherein the condition is recessive dystrophic epidermolysis bullosa, and
      wherein within about 12 weeks of the administering, a lesion in an area of skin associated with the recessive dystrophic epidermolysis bullosa resolves as determined by magnetic resonance imaging of the area of skin.

### BRIEF DESCRIPTION OF THE DRAWINGS

FIG. 1 Rigosertib induces cell death in RDEB SCC keratinocytes without affecting normal primary keratinocytes. Panel A. Cells were seeded at high density and exposed to Rigosertib or vehicle control for 48 hours after which cells were fixed and stained with crystal violet. Panel B. Phase contrast images of unfixed cells in culture after given treatment of either control (left) or Rigosertib (1 μM, right).

FIG. 2 Rigosertib inhibits RDEB SCC keratinocyte grown in vivo. SCC RDEB16 keratinocytes were inoculated subcutaneously to Balb/c SCID mice. After tumors reached 100 mm³, vehicle control, Rigosertib or Compound 1 (ATP-competitive inhibitor) were administered 6 times and tumor volume was measured with calipers. Tumors were harvested and photographed at day 35.

FIG. 3 shows clinical photography images of target legions on the left hand (Panels 3A, 3C, 3E, 3G, and 3I) and the right elbow (Panels 3B, 3D, 3F, 3H, and 3J) at V1 (day 1), V13 (days 85-95), V25 (day 169-175), V41 (days 281-283), and V52 (d358-364). At V25, both lesions were biopsied, with no histological signs of malignancy.

### DETAILED DESCRIPTION

Ras is a frequently mutated gene causing cancer. A significant number of patients with non-small cell lung cancer (NSCLC) have a mutation of RAS, most frequently KRAS. In some embodiments, a mechanism of action for a compound disclosed herein, for example, rigosertib, is to down-modulate the mutated RAS pathway. This modulation of the RAS pathway can result in less production of ERK, a protein involved in cell proliferation. When ERK is overexpressed, ERK can provide a proliferative advantage to cells, thus leading to cancer. KRAS-mutated NSCLC can be managed with, for example, a checkpoint inhibitor.

Rigosertib ((E)-2-(5-((2,4,6-trimethoxystyrylsulfonyl)methyl)-2-methoxyphenylamino)acetic acid, or a pharmaceutically-acceptable salt or zwitterion thereof) can block the RAS cascade and promote the expression of novel antigens on the tumor's surface. This process can turn cold tumors that are not surrounded by host lymphocytes into hot tumors that are surrounded by host lymphocytes. Ibis modulation of the tumor micro-environment (TME) in combination with the checkpoint blockade can facilitate the host immune system to contribute to tumor control. The host lymphocytes can contribute to tumor control when exposed to a checkpoint inhibitor.

The present disclosure provides a method of treating a condition in a subject in need thereof, the method comprising administering to the subject a therapeutically-effective amount of a compound of the formula:

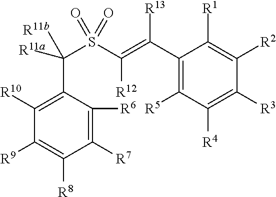

or a pharmaceutically-acceptable salt or zwitterion thereof,
wherein:
each R¹, R², R³, R⁴, R⁵, R⁶, R⁷, R⁸, R⁹, R¹⁰, R^{11a}, R^{11b}, R¹², and R¹³ is independently alkyl, alkenyl, alkynyl, alkoxy, aryl, heteroaryl, heterocyclyl, C(O)R^{x}, C(O)OR^{x}, C(O)NR^{x}R^{y}, OR^{x}, SR^{x}, NR^{x}R^{y}, NR^{x}C(O)R^{y}, OC(O)R^{x}, or SiR^{x}R^{y}R^{z}, each of which is independently substituted or unsubstituted; or hydrogen or halogen; and
each R^{x}, R^{y}, and R^{z} is independently alkyl, alkenyl, alkynyl, aryl, heteroaryl, or heterocyclyl, each of which is independently substituted or unsubstituted; or hydrogen or halogen,
   wherein the condition is recessive dystrophic epidermolysis bullosa, and
   wherein within about 12 weeks of the administering, a lesion in an area of skin associated with the recessive dystrophic epidermolysis bullosa resolves as determined by magnetic resonance imaging of the area of skin.

The present disclosure also provides a method of treating a squamous cell carcinoma, comprising administering to a subject in need thereof a therapeutically-effective amount of a pharmaceutical composition, the pharmaceutical composition comprising in a unit dosage form a compound of the disclosure, for example, (E)-2-(5-((2,4,6-trimethoxystyrylsulfonyl)methyl)-2-methoxyphenylamino)acetic acid, or a pharmaceutically-acceptable salt or zwitterion thereof.

The present disclosure further provides a method of treating a subject having recessive dystrophic epidermolysis bullosa (RDEB)-associated locally advanced/metastatic squamous cell carcinoma (SCC), the method comprising administering to the subject in need thereof a therapeutically-effective amount of a pharmaceutical composition, the pharmaceutical composition comprising in a unit dosage form a compound of the disclosure, for example, (E)-2-(5-((2,4,6-trimethoxystyrylsulfonyl)methyl)-2-methoxyphenylamino)acetic acid, or a pharmaceutically-acceptable salt or zwitterion thereof.

### Compounds of the Disclosure

In some embodiments, disclosed herein is a compound of the formula:

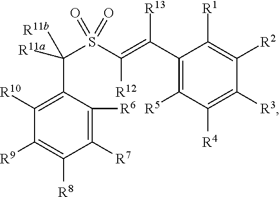

wherein:
each R¹, R², R³, R⁴, R⁵, R⁶, R⁷, R⁸, R⁹, R¹⁰, R^{11a}, R^{11b}, R¹², and R¹³ is independently alkyl, alkenyl, alkynyl, alkoxy, aryl, heteroaryl, heterocyclyl, C(O)R^{x}, C(O)NR^{x}R^{y}, OR^{x}, SR^{x}, NR^{x}R^{y}, NR^{x}C(O)R^{y}, OC(O)R^{x}, or SiR^{x}R^{y}Z, each of which is independently substituted or unsubstituted; or hydrogen or halogen; and
each R^{x}, R^{y}, and R^{z} is independently alkyl, alkenyl, alkynyl, aryl, heteroaryl, or heterocyclyl, each of which is independently substituted or unsubstituted; or hydrogen or halogen,
   or a pharmaceutically-acceptable salt or zwitterion thereof.

In some embodiments, R¹, R³, and R⁵ are the same. In some embodiments, R¹, R³, R⁵, and R⁸ are the same. In some embodiments, each R¹, R³, and R⁵ is independently OR^{x}. In some embodiments, each R¹, R³, R⁵, and R⁸ is independently OR^{x}. In some embodiments, each R^{x} is independently alkyl, aryl, heteroaryl, or heterocyclyl, each of which is independently substituted or unsubstituted. In some embodiments, each R^{x} is hydrogen. In some embodiments, each R^{x} is independently unsubstituted C₁₋₆ alkyl. In some embodiments, each R^{x} is independently unsubstituted C₁₋₃ alkyl. In some embodiments, each R^{x} is methyl. In some embodiments, each R^{x} is ethyl. In some embodiments, each R^{x} is independently substituted C₁₋₆ alkyl. In some embodiments, each R^{x} is independently substituted C₁₋₃ alkyl. In some embodiments, each R^{x} is C₁ alkyl substituted with hydroxy, sulfhydryl, halogen, an amino group, a nitro group, cyano, a sulfoxide group, a sulfone group, a sulfonamide group, a carboxyl group, a carboxylic acid, a carboxaldehyde group, alkoxy, aryl, heterocyclyl, acyl, amide, or ester.

In some embodiments, R² is hydrogen. In some embodiments, R⁴ is hydrogen. In some embodiments, R⁶ is hydrogen. In some embodiments, R⁹ is hydrogen. In some embodiments, R¹⁰ is hydrogen.

In some embodiments, R⁷ is alkyl, alkoxy, aryl, heteroaryl, heterocyclyl, OR^{x}, or NR^{x}R^{y}. In some embodiments, R⁷ is NR^{x}R^{y}. In some embodiments, R^{x} is hydrogen. In some embodiments, R^{y} is hydrogen. In some embodiments, R^{y} is substituted alkyl. In some embodiments, R^{y} is substituted C₁₋₆ alkyl. In some embodiments, R^{y} is C₁ alkyl substituted with hydroxy, sulfhydryl, halogen, an amino group, a nitro group, cyano, a sulfoxide group, a sulfone group, a sulfonamide group, a carboxyl group, a carboxylic acid, a carboxaldehyde group, alkoxy, aryl, a heterocyclyl group, an acyl group, amide, or an ester. In some embodiments, R^{y} is CH₂COOH.

In some embodiments, each R^{11a} and R^{11b} is independently alkyl, aryl, heteroaryl, or heterocyclyl, each of which is independently substituted or unsubstituted. In some embodiments, each R^{11a} and R^{11b} is independently substituted C₁₋₆ alkyl. In some embodiments, each R^{11a} and R^{11b} is independently unsubstituted C₁₋₆ alkyl. In some embodiments, each R¹² and R¹³ is independently alkyl, aryl, heteroaryl, or heterocyclyl, each of which is independently substituted or unsubstituted. In some embodiments, each R¹² and R¹³ is independently substituted C₁₋₆ alkyl. In some embodiments, each R¹² and R¹³ is independently unsubstituted C₁₋₆ alkyl. In some embodiments, R^{11a} is hydrogen. In some embodiments, R^{11b} is hydrogen. In some embodiments, R¹² is hydrogen. In some embodiments, R¹³ is hydrogen.

In some embodiments, disclosed herein is a compound of the formula:

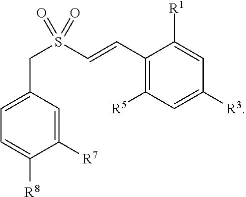

In some embodiments, the compound has the formula:

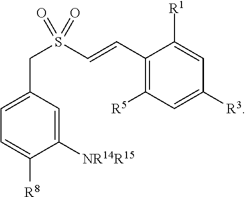

In some embodiments, each R¹, R³, R⁵, and R⁸ is independently OR^{x}. In some embodiments, each R^{x} is independently alkyl, aryl, heteroaryl, heterocyclyl, each of which is independently substituted or unsubstituted. In some embodiments, each R^{x} is independently hydrogen. In some embodiments, each R^{x} is independently unsubstituted C₁₋₆ alkyl. In some embodiments, each R^{x} is independently unsubstituted C₁₋₃ alkyl. In some embodiments, each R^{x} is independently methyl. In some embodiments, each R^{x} is independently ethyl. In some embodiments, each R^{x} is independently substituted C₁₋₆ alkyl. In some embodiments, each R^{x} is independently substituted C₁₋₃ alkyl. In some embodiments, each R^{x} is independently methyl that is substituted.

In some embodiments, each R¹⁴ and R¹⁵ is independently alkyl, alkoxy, aryl, heteroaryl, or heterocyclyl, each of which is independently substituted or unsubstituted; or hydrogen. In some embodiments, R¹⁴ is H. In some embodiments, R¹⁵ is H. In some embodiments, R¹⁵ is substituted alkyl. In some embodiments, R¹⁵ is substituted C₁₋₆ alkyl. In some embodiments, R¹⁵ is substituted C₁ alkyl. In some embodiments, R¹⁵ is CH₂COOH.

In some embodiments, the compound has the formula:

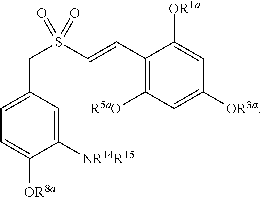

In some embodiments, each R^{1a}, R^{3a}, R^{5a}, and R^{8a} is the same. In some embodiments, each R^{1a}, R^{3a}, R^{5a}, and R^{8a} is different. In some embodiments, each R^{1a}, R^{3a}, R^{5a}, and R^{8a} is independently substituted or unsubstituted alkyl. In some embodiments, each R^{1a}, R^{3a}, R^{5a}, and R^{8a} is independently substituted or unsubstituted C₁₋₈ alkyl. In some embodiments, each R^{1a}, R^{3a}, R^{5a}, and R^{8a} is independently substituted or unsubstituted C₁₋₃ alkyl. In some embodiments, each Ria, R^{3a}, R^{5a}, and R^{8a} is independently substituted or unsubstituted C₁ alkyl. In some embodiments, each R^{1a}, R^{3a}, R^{5a}, and R^{8a} is independently methyl. In some embodiments, each R^{1a}, R^{3a}, R^{5a}, and R^{8a} is independently ethyl.

In some embodiments, each R¹⁴ and R¹⁵ is independently alkyl, alkoxy, aryl, heteroaryl, heterocyclyl, each of which is independently substituted or unsubstituted; or hydrogen. In some embodiments, R¹⁴ is H. In some embodiments, R¹⁵ is H. In some embodiments, R¹⁵ is substituted alkyl. In some embodiments, R¹⁵ is substituted C₁₋₆ alkyl. In some embodiments, R¹⁵ is substituted C₁ alkyl. In some embodiments, R¹⁵ is CH₂COOH.

In some embodiments, the compound has the formula:

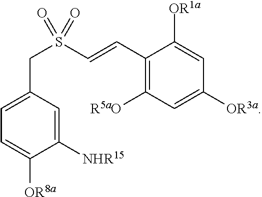

In some embodiments, each R^{1a}, R^{3a}, R^{5a}, and R^{8a} is independently substituted or unsubstituted alkyl. In some embodiments, each R^{1a}, R^{3a}, R^{5a}, and R^{8a} is independently substituted or unsubstituted C₁₋₈ alkyl. In some embodiments, R^{1a} is C₁₋₆ alkyl. In some embodiments, R^{3a} is C₁₋₆ alkyl. In some embodiments, R^{5a} is C₁₋₆ alkyl. In some embodiments, R^{8a} is C₁₋₆ alkyl. In some embodiments, R^{1a} is methyl. In some embodiments, R^{3a} is methyl. In some embodiments, R^{5a} is methyl. In some embodiments, R^{8a} is methyl.

In some embodiments, R¹⁵ is substituted alkyl. In some embodiments, R¹⁵ is substituted C₁₋₆ alkyl. In some embodiments, R¹⁵ is substituted C₁ alkyl. In some embodiments, R¹⁵ is CH₂COOH.

In some embodiments, disclosed herein is a compound of the formula:

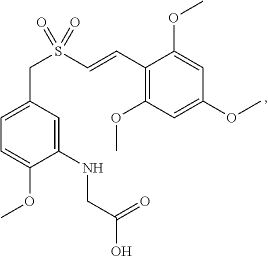 [SMILES: COc1cc(OC)c(/C=C/S(=O)(=O)Cc2ccc(OC)c(NCC(=O)O)c2)c(OC)c1]

(E)-2-(5-((2,4,6-trimethoxystyrylsulfonyl)methyl)-2-methoxyphenylamino)acetic acid, or a pharmaceutically-acceptable salt or zwitterion thereof. In some embodiments, the compound is

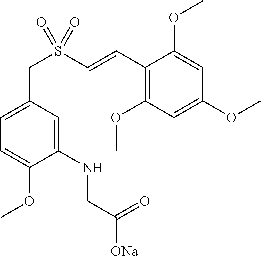

sodium (E)-2-(5-((2,4,6-trimethoxystyrylsulfonyl)methyl)-2-methoxyphenylamino)acetate. In some embodiments, a compound disclosed herein is a sodium salt.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups.

Non-limiting examples of alkyl and alkylene groups include straight, branched, and cyclic alkyl and alkylene groups. An alkyl or alkylene group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. In some embodiments, alkyl or alkylene is C₁₋₈ alkyl or C₁₋₈ alkylene that is substituted or unsubstituted. In some embodiments, alkyl or alkylene is C₁₋₆ alkyl or C₁₋₆ alkylene that is substituted or unsubstituted. In some embodiments, alkyl or alkylene is C₁₋₃ alkyl or C₁₋₃ alkylene that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl or alkynylene groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl. Non-limiting examples of substituted aryl groups include 3,4-dimethylphenyl, 4-tert-butylphenyl, 4-cyclopropylphenyl, 4-diethylaminophenyl, 4-(trifluoromethyl)phenyl, 4-(difluoromethoxy)-phenyl, 4-(trifluoromethoxy)phenyl, 3-chlorophenyl, 4-chlorophenyl, 3,4-dichlorophenyl, 2-fluorophenyl, 2-chlorophenyl, 2-iodophenyl, 3-iodophenyl, 4-iodophenyl, 2-methylphenyl, 3-fluorophenyl, 3-methylphenyl, 3-methoxyphenyl, 4-fluorophenyl, 4-methylphenyl, 4-methoxyphenyl, 2,3-difluorophenyl, 3,4-difluorophenyl, 3,5-difluorophenyl, 2,3-dichlorophenyl, 3,4-dichlorophenyl, 3,5-dichlorophenyl, 2-hydroxyphenyl, 3-hydroxyphenyl, 4-hydroxyphenyl, 2-methoxyphenyl, 3-methoxyphenyl, 4-methoxyphenyl, 2,3-dimethoxyphenyl, 3,4-dimethoxyphenyl, 3,5-dimethoxyphenyl, 2,4-difluorophenyl, 2,5-difluorophenyl, 2,6-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 2,4-dichlorophenyl, 2,5-dichlorophenyl, 2,6-dichlorophenyl, 3,4-dichlorophenyl, 2,3,4-trichlorophenyl, 2,3,5-trichlorophenyl, 2,3,6-trichlorophenyl, 2,4,5-trichlorophenyl, 3,4,5-trichlorophenyl, 2,4,6-trichlorophenyl, 2,3-dimethylphenyl, 2,4-dimethylphenyl, 2,5-dimethylphenyl, 2,6-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 2,3,4-triethylphenyl, 2,3,5-triethylphenyl, 2,3,6-triethylphenyl, 2,4,5-triethylphenyl, 2,4,6-triethylphenyl, 2-isopropylphenyl, 3-isopropylphenyl, and 4-isopropylphenyl.

Non-limiting examples of substituted aryl groups include 2-aminophenyl, 2-(N-methylamino)phenyl, 2-(N,N-dimethylamino)phenyl, 2-(N-ethylamino)phenyl, 2-(N,N-diethylamino)phenyl, 3-aminophenyl, 3-(N-methylamino)phenyl, 3-(N,N-dimethylamino)phenyl, 3-(N-ethylamino)phenyl, 3-(N,N-diethylamino)phenyl, 4-aminophenyl, 4-(N-methylamino)phenyl, 4-(N,N-dimethylamino)phenyl, 4-(N-ethylamino)phenyl, and 4-(N,N-diethylamino)phenyl.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

Non-limiting examples of heterocycles include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

In some embodiments, the compound is at least about 85% pure. In some embodiments, the compound is at least about 90% pure. In some embodiments, the compound is at least about 95% pure. In some embodiments, the compound is at least about 98% pure. In some embodiments, the compound is at least about 99% pure. In some embodiments, the compound is at least about 99.5% pure.

### Pharmaceutically Acceptable Salts

The method disclosed herein provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is a sodium salt.

Metal salts can arise from the addition of an inorganic base to a compound disclosed herein. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Acid addition salts can arise from the addition of an acid to a compound disclosed herein. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

### Pharmaceutical Compositions

A pharmaceutical composition of the invention can be used, for example, before, during, or after treatment of a subject with, for example, another pharmaceutical agent.

Subjects can be, for example, elderly adults, adults, adolescents, pre-adolescents, children, toddlers, infants, neonates, and non-human animals. In some embodiments, a subject is a patient.

A pharmaceutical composition of the invention can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration. Pharmaceutical compositions can be administered in therapeutically-effective amounts as pharmaceutical compositions by various forms and routes including, for example, intravenous, intravitreal, intranasal, inhalation, nasal inhalation, mouth inhalation, intratracheal, intrapulmonary, transmucosal, subcutaneous, intramuscular, oral, rectal, aerosol, parenteral, ophthalmic, pulmonary, transdermal, vaginal, otic, nasal, and topical administration.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

In some embodiments, a composition of the disclosure can be formulated for oral administration. In some embodiments, a compound of the disclosure can be formulated as a capsule. In some embodiments, a compound of the disclosure can be formulated as a soft gelatin capsule. In some embodiments, a compound of the disclosure can be formulated to comprise a compound of the disclosure at a concentration of at least about 20 mg/mL, at least about 30 mg/mL, about 40 mg/mL, about 50 mg/mL, about 60 mg/mL, at least about 70 mg/mL, at least about 80 mg/mL, at least about 90 mg/mL, at least about 100 mg/mL, at least about 120 mg/mL, at least about 140 mg/mL, at least about 160 mg/mL, at least about 180 mg/mL, at least about 200 mg/mL, at least about 220 mg/mL, at least about 240 mg/mL, at least about 260 mg/mL, at least about 280 mg/mL, at least about 300 mg/mL, at least about 320 mg/mL, at least about 340 mg/mL, at least about 360 mg/mL, at least about 380 mg/mL, at least about 400 mg/mL, at least about 420 mg/mL, at least about 440 mg/mL, at least about 460 mg/mL, at least about 480 mg/mL, or at least about 500 mg/mL. In some embodiments, a compound of the disclosure can be formulated to comprise a compound of the disclosure at a concentration of about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 120 mg/mL, about 140 mg/mL, about 160 mg/mL, about 180 mg/mL, about 200 mg/mL, about 220 mg/mL, about 240 mg/mL, about 260 mg/mL, about 280 mg/mL, about 300 mg/mL, about 320 mg/mL, about 340 mg/mL, about 360 mg/mL, about 380 mg/mL, about 400 mg/mL, about 420 mg/mL, about 440 mg/mL, about 460 mg/mL, about 480 mg/mL, or about 500 mg/mL. In some embodiments, a compound of the disclosure can be formulated to comprise a compound of the disclosure at a concentration of at most about 20 mg/mL, at most about 30 mg/mL, about 40 mg/mL, about 50 mg/mL, about 60 mg/mL, at most about 70 mg/mL, at most about 80 mg/mL, at most about 90 mg/mL, at most about 100 mg/mL, at most about 120 mg/mL, at most about 140 mg/mL, at most about 160 mg/mL, at most about 180 mg/mL, at most about 200 mg/mL, at most about 220 mg/mL, at most about 240 mg/mL, at most about 260 mg/mL, at most about 280 mg/mL, at most about 300 mg/mL, at most about 320 mg/mL, at most about 340 mg/mL, at most about 360 mg/mL, at most about 380 mg/mL, at most about 400 mg/mL, at most about 420 mg/mL, at most about 440 mg/mL, at most about 460 mg/mL, at most about 480 mg/mL, or at most about 500 mg/mL. In some embodiments, a compound of the disclosure can be formulated to comprise a compound of the disclosure at a concentration of about 70 mg/mL. In some embodiments, a compound of the disclosure can be formulated to comprise a compound of the disclosure at a concentration of about 100 mg/mL. In some embodiments, a compound of the disclosure can be formulated to comprise a compound of the disclosure at a concentration of about 150 mg/mL. In some embodiments, a compound of the disclosure can be formulated to comprise a compound of the disclosure at a concentration of about 200 mg/mL. In some embodiments, a compound of the disclosure can be formulated to comprise a compound of the disclosure at a concentration of about 250 mg/mL. In some embodiments, a compound of the disclosure can be formulated to comprise a compound of the disclosure at a concentration of about 280 mg/mL.

In some embodiments, a compound of the disclosure is formulated for oral administration in a suspension of polyethylene glycol (PEG). In some embodiments, a compound of the disclosure is formulated for oral administration in a suspension of PEG 400. In some embodiments, a compound of the disclosure is formulated for oral administration in a suspension of PEG 4000. In some embodiments, a compound of the disclosure is formulated for oral administration in a suspension of PEG 400 and PEG 4000.

In some embodiments, a capsule formulated for oral administration can comprise at least about 0.1 mL, at least about 0.2 mL, at least about 0.3 mL, at least about 0.4 mL, at least about 0.5 mL, at least about 0.6 mL, at least about 0.7 mL, at least about 0.8 mL, at least about 0.9 mL, at least about 1 mL, at least about 1.1 mL, at least about 1.2 mL, at least about 1.3 mL, at least about 1.4 mL, or at least about 1.5 mL of a solution comprising a compound of the disclosure. In some embodiments, a capsule formulated for oral administration can comprise about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, or about 1.5 mL of a solution comprising a compound of the disclosure. In some embodiments, a capsule formulated for oral administration can comprise at most about 0.1 mL, at most about 0.2 mL, at most about 0.3 mL, at most about 0.4 mL, at most about 0.5 mL, at most about 0.6 mL, at most about 0.7 mL, at most about 0.8 mL, at most about 0.9 mL, at most about 1 mL, at most about 1.1 mL, at most about 1.2 mL, at most about 1.3 mL, at most about 1.4 mL, or at most about 1.5 mL of a solution comprising a compound of the disclosure. In some embodiments, a capsule formulated for oral administration can comprise at most about 0.5 mL of a solution comprising a compound of the disclosure. In some embodiments, a capsule formulated for oral administration can comprise at most about 0.8 mL of a solution comprising a compound of the disclosure. In some embodiments, a capsule formulated for oral administration can comprise at most about 1 mL of a solution comprising a compound of the disclosure. In some embodiments, a capsule formulated for oral administration can comprise at most about 1.2 mL of a solution comprising a compound of the disclosure. In some embodiments, a capsule formulated for oral administration can comprise at most about 1.5 mL of a solution comprising a compound of the disclosure.

In some embodiments, a capsule for oral administration can be clear or transparent. In some embodiments, a capsule for oral administration can be opaque. In some embodiments, a capsule for oral administration can be opaque and yellow or orange in color.

For oral administration, pharmaceutical compositions can be formulated readily by combining the active compounds with pharmaceutically-acceptable carriers or excipients. Such carriers can be used to formulate tablets, powders, pills, dragees, capsules, liquids, gels, syrups, elixirs, slurries, or suspensions for oral ingestion by a subject.

Pharmaceutical preparations for oral use can be obtained by mixing one or more solid excipient with one or more compounds described herein, optionally grinding the resulting mixture, and processing the mixture of granules, after adding suitable auxiliaries, if desired, to obtain tablets or dragee cores. Cores can be provided with suitable coatings. For this purpose, concentrated sugar solutions can be used, which can contain an excipient such as gum arabic, talc, polyvinylpyrrolidone, carbopol gel, polyethylene glycol, or titanium dioxide, lacquer solutions, and suitable organic solvents or solvent mixtures. Dyestuffs or pigments can be added to the tablets or dragee coatings, for example, for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. A gelatin can be alkaline-processed. The push-fit capsules can contain the active ingredients in admixture with filler such as lactose, binders such as starches, or lubricants such as talc or magnesium stearate, and stabilizers. In soft capsules, the active compounds can be dissolved or suspended in suitable liquids, such as fatty oils, liquid paraffin, or liquid polyethylene glycols. Stabilizers can be added. All formulations for oral administration are provided in dosages suitable for such administration.

For buccal or sublingual administration, the compositions can be tablets, lozenges, or gels.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

A pharmaceutical formulation disclosed herein can be, for example, a parenteral formulation of a solution of the active pharmaceutical ingredient (API). In some embodiments, a compound of the disclosure can be prepared for intravenous administration at a concentration of from about 25 mg/mL to about 50 mg/mL, from about 50 mg/mL to about 75 mg/mL, from about 75 mg/mL to about 100 mg/mL, from about 100 mg/mL to about 125 mg/mL, from about 125 mg/mL to about 150 mg/mL, from about 150 mg/mL to about 175 mg/mL, from about 175 mg/mL to about 200 mg/mL, from about 200 mg/mL to about 225 mg/mL, or from about 225 mg/mL to about 250 mg/mL in a liquid vehicle. In some embodiments, a compound of the disclosure can be prepared for intravenous administration at a concentration of at least about 25 mg/mL, at least about 50 mg/mL, at least about 75 mg/mL, at least about 100 mg/mL, at least about 125 mg/mL, at least about 150 mg/mL, at least about 175 mg/mL, at least about 200 mg/mL, at least about 225 mg/mL, or at least about 250 mg/mL in a liquid vehicle. In some embodiments, a compound of the disclosure can be prepared for intravenous administration at a concentration of about 25 mg/mL, about 50 mg/mL, about 75 mg/mL, about 100 mg/mL, about 125 mg/mL, about 150 mg/mL, about 175 mg/mL, about 200 mg/mL, about 225 mg/mL, or about 250 mg/mL in a liquid vehicle. In some embodiments, a compound of the disclosure can be prepared for intravenous administration at a concentration of at most about 25 mg/mL, at most about 50 mg/mL, at most about 75 mg/mL, at most about 100 mg/mL, at most about 125 mg/mL, at most about 150 mg/mL, at most about 175 mg/mL, at most about 200 mg/mL, at most about 225 mg/mL, or at most about 250 mg/mL in a liquid vehicle. In some embodiments, the liquid vehicle is an organic solvent. In some embodiments, the liquid vehicle is polyethylene glycol (PEG). In some embodiments, the liquid vehicle is PEG 400. In some embodiments, the liquid vehicle is adjusted to pH 7-13 or pH 12-13 using a sodium hydroxide (NaOH) solution. In some embodiments, a pharmaceutical composition for intravenous administration can comprise a compound of the disclosure at a concentration of about 75 mg/mL in a liquid vehicle of polyethylene glycol (PEG) 400 with pH 7-13 or pH 12-13 adjusted by use of sodium hydroxide (NaOH) solution. The solution can be a clear, colorless to pale yellow, sterile, preservative-free solution packaged in a clear glass vial sealed with a Teflon-coated rubber stopper. A vial disclosed herein can be a 30-mL clear glass vial containing, for example, 24 mL of the parenteral formulation. Dilution with IV saline can be required when the parenteral formulation is administered IV.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives. A pharmaceutical composition of a compound disclosed herein can be a combination of any pharmaceutical compounds described herein with other chemical components, such as carriers, stabilizers, diluents, dispersing agents, suspending agents, thickening agents, or excipients. The pharmaceutical composition facilitates administration of the compound to an organism.

The compounds of the invention can be applied topically to the skin, or a body cavity, for example, oral, vaginal, bladder, cranial, spinal, thoracic, or pelvic cavity of a subject. The compounds of the invention can be applied to an accessible body cavity.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, and PEG. In suppository forms of the compositions, a low-melting wax such as a mixture of fatty acid glycerides, optionally in combination with cocoa butter, can be melted.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives. The methods and pharmaceutical compositions described herein include the use of crystalline forms (also known as polymorphs), and active metabolites of these compounds having the same type of activity.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include liquid, powder, gel, nanosuspension, nanoparticle, microgel, aqueous or oily suspensions, emulsion, and any combination thereof. Non-limiting examples of dosage forms suitable for use in a method disclosed herein include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof. Non-limiting examples of pharmaceutically-acceptable excipients suitable for use in the method disclosed herein include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, anti-microbial agents, plant cellulosic material and spheronization agents, and any combination thereof.

A composition of the invention can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Compounds can be delivered via liposomal technology. The use of liposomes as drug carriers can increase the therapeutic index of the compounds. Liposomes are composed of natural phospholipids, and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellar vesicle (SUV), and the large unilamellar vesicle (LUV). Liposomal physicochemical properties can be modulated to optimize penetration through biological barriers and retention at the site of administration, and to reduce a likelihood of developing premature degradation and toxicity to non-target tissues. Optimal liposomal properties depend on the administration route: large-sized liposomes show good retention upon local injection, small-sized liposomes are better suited to achieve passive targeting. PEGylation reduces the uptake of the liposomes by the liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, granulating, dragee-making, levigating, emulsifying, encapsulating, entrapping, or compression processes.

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Dosing.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A compound described herein can be present in a composition in a range of from about 1 mg to about 2000 mg; from about 100 mg to about 2000 mg; from about 10 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2100 mg, about 2200 mg, about 2300 mg, about 2400 mg, or about 2500 mg.

In some embodiments, a dose can be expressed in terms of an amount of the drug divided by the mass of the subject, for example, milligrams of drug per kilograms of subject body mass. In some embodiments, a compound is administered in an amount ranging from about 5 mg/kg to about 50 mg/kg, 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg.

### Methods of Treatment

In some embodiments, compounds of the invention can be used to treat cancer in a subject. A compound of the invention can, for example, slow the proliferation of cancer cell lines, or kill cancer cells. Non-limiting examples of cancer that can be treated by a compound of the invention include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor.

A method disclosed herein can be used to treat, for example, a proliferative disease, a cancer, a cancer metastasis, a solid tumor, a liquid tumor, a carcinoma, a squamous cell carcinoma (SCC), a SCC associated with a disease, for example, recessive dystrophic epidermolysis bullosa (RDEB), or any metastases thereof.

A tumor response due to a method disclosed herein can be measured based on the RECIST classification of responses.

Recessive dystrophic epidermolysis bullosa (RDEB or EB) is a severe genodermatosis caused by mutations in COL7A1, characterized by generalized skin blistering and involvement of mucous membranes. Aggressive metastasizing squamous cell carcinomas (SCCs) typically arising in areas of chronic skin wounding are a common complication that can significantly contribute to the reduced patient average life expectancy of less than 40 years. Individuals can develop highly aggressive early onset tumors often arising multiply at different sites in individuals age 20 to 40. These neoplasms can show limited response rates of mostly short duration to conventional chemo- and radiotherapy. Moreover, tolerability of aggressive cytotoxic chemotherapy (e.g. as for skin, mucosal, renal as well as bone marrow toxicity with neutropenia and risk of septicaemia) is poor along with EB-associated disease burden and systemic morbidity.

Squamous cell carcinoma is a carcinoma that occur in many different organs, including the skin, mouth, esophagus, lungs, and cervix. This cancer is a malignant tumor of epithelium that shows squamous cell differentiation. Squamous cell carcinoma is usually developed in the epithelial layer of the skin and sometimes in various mucous membranes of the body. This type of cancer can be seen on the skin, lips, inside the mouth, throat or esophagus.

RDEB is an inherited blistering skin disorder that is associated with significant esophageal strictures, resulting in dysphagia and nutritional failure. Esophageal dysphagia refers to the sensation of food sticking or getting caught in the base of the throat or in the chest after a subject starts swallowing food or drink. Esophageal stricture refers to a narrowed esophagus that can trap large pieces of food. Scar tissue can cause narrowing of the esophagus. In some embodiments, a subject administered with a therapeutically-effective amount of a compound of the disclosure has an esophageal obstruction.

Provided herein is a method for treating squamous cell carcinoma (SCC) by administering a compound of the disclosure. Provided herein is a method for treating metastasizing SCC by administering a compound of the disclosure. Provided herein is a method for treating aggressive metastasizing SCC by administering a compound of the disclosure.

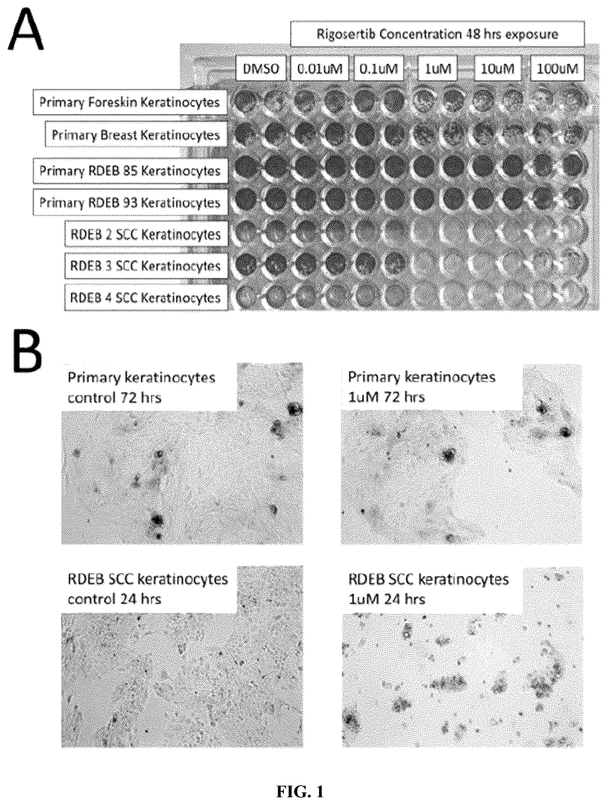

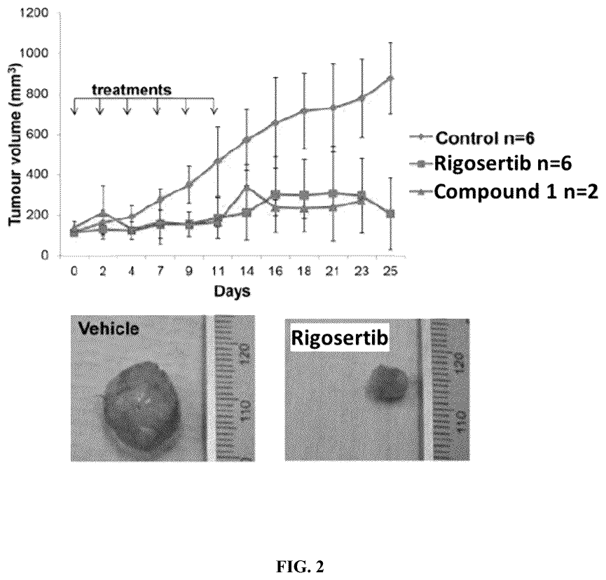

As demonstrated herein, RDEB SCC keratinocytes are sensitive to a compound of the disclosure at concentrations lower than that needed for an equivalent response in normal keratinocytes or fibroblasts isolated from RDEB patients (FIG. 1). Efficacy of a compound of the disclosure for treating RDEB SCC in tumor xenograft experiments is shown in FIG. 2.

In some embodiments, disclosed herein is a method of assaying for a biomarker to indicate presence of a condition, and administering a compound of pharmaceutical composition of the disclosure. In some embodiments, the biomarker is a genetic biomarker. In some embodiments, the biomarker is a protein biomarker. In some embodiments, ASSAY, BIOLOGICAL SAMPLE, METHODS.

In some embodiments, disclosed herein is a method of assaying a subject for a biomarker of a phosphatidylinositol-3-kinase (PI3K) pathway prior to administering a compound of the disclosure. In some embodiments, the PI3K pathway biomarker is a mutation in the PIK3CA gene. In some embodiments, the PI3K pathway biomarker is a mutation in PTEN loss. In some embodiments, the PI3K pathway biomarker is p4E-BP1 or pAkt. In some embodiments, the PI3K pathway biomarker is p21-activated kinase 1 (PAK1) protein.

In some embodiments, disclosed herein is a method of assaying a subject for a biomarker of a protein kinase B (Akt) pathway prior to administering a compound of the disclosure. In some embodiments, the Akt pathway biomarker is the PIKCA gene. In some embodiments, the Akt pathway biomarker is PTEN loss. In some embodiments, the Akt pathway biomarker is Bcl2-related death protein (BAD). In some embodiments, the Akt pathway biomarker is phosphatidylinositol 3,4,5-triphosphate (PIP3).

In some embodiments, disclosed herein is a method of assaying a subject for a biomarker of a serine/threonine-protein kinase 1 (PLKT) pathway prior to administering a compound of the disclosure. In some embodiments, the PLK1 pathway biomarker is a mutation in a splicing factor (SF). In some embodiments, the PLK1 pathway biomarker is SRSF2. In some embodiments, the PLK1 pathway biomarker is SF3BT. In some embodiments, the PLK1 pathway biomarker is centrosomal protein 55 kDa (CEP55).

### Dosing

A dosing regimen disclosed herein can be, for example, one oral dose of a compound of the disclosure in the morning, and a second dose of the compound in the afternoon or evening. In some embodiments, the morning dose and the afternoon or evening dose can be the same. In some embodiments, the morning dose and the afternoon or evening dose can be different. In some embodiments, one oral dose of 840 mg of rigosertib can be administered in the morning, and a 560 mg oral dose of rigosertib can be administered in the afternoon.

In some embodiments, a dose can be escalated depending on the observed number of dose limiting toxicities (DLTs). In some embodiments, dose escalation can continue in about 70 mg, about 140 mg, about 280 mg, about 560 mg, or about 1120 mg increments. In some embodiments, dose escalation can continue in 280 mg increments. In some embodiments, dose escalation can continue in 560 mg increments. In some embodiments, a morning dose can be increased by 280 mg increments, and an afternoon dose can be increased by 280 mg increments. In some embodiments, a morning dose can be increased by 560 mg increments, and an afternoon dose can be increased by 560 mg increments.

A dose escalation can continue until two or more DLTs are observed in a single cohort. At that point, a prior dose cohort can be expanded to 6 patients and if less than 2 DLTs occur in those 6 patients, then that dose can be considered the MTD. Additionally, a much larger percentage of the total daily dose can be administered in the morning dose and a lower percentage in the afternoon dose (i.e. 1,400 mg in the a.m. and 560 mg in the afternoon).

A dosing regimen for intravenous rigosertib can be, for example, at least about 300 mg/day, at least about 600 mg/day, at least about 900 mg/day, at least about 1200 mg/day, at least about 1500 mg/day, at least about 1800 mg/day, at least about 2100 mg/day, at least about 2400 mg/day, at least about 2700 mg/day, or at least about 3000 mg/day. In some embodiments, a dosing regimen for intravenous rigosertib can be, for example, about 300 mg/day, about 600 mg/day, about 900 mg/day, about 1200 mg/day, about 1500 mg/day, about 1800 mg/day, about 2100 mg/day, about 2400 mg/day, about 2700 mg/day, or about 3000 mg/day. In some embodiments, a dosing regimen for intravenous rigosertib can be, for example, at most about 300 mg/day, at most about 600 mg/day, at most about 900 mg/day, at most about 1200 mg/day, at most about 1500 mg/day, at most about 1800 mg/day, at most about 2100 mg/day, at most about 2400 mg/day, at most about 2700 mg/day, or at most about 3000 mg/day. In some embodiments, a dosing regimen for intravenous rigosertib can be about 1800 mg/day. In some embodiments, a dosing regimen for intravenous rigosertib can be about 2100 mg/day. In some embodiments, a dosing regimen for intravenous rigosertib can be about 2400 mg/day.

In some embodiments, a dosing regimen for intravenous rigosertib can comprise administration on days 1-3 of a 14-day cycle. In some embodiments, a dosing regimen for intravenous rigosertib can comprise administration on days 1-3 of a 28-day cycle. In some embodiments, a dosing regimen for intravenous rigosertib can comprise administration on days 1-3 of a 14-day cycle, then on days 1-3 of a 28-day cycle thereafter. In some embodiments, a dosing regimen for intravenous rigosertib can comprise increasing doses on days 1-3 of a 14 day cycle. In some embodiments, a dosing regimen for intravenous rigosertib can comprise increasing doses on days 1-3 of a 28 day cycle.

In some embodiments, a dosing regimen for intravenous rigosertib can comprise administration on days 1-3 of a 14-day cycle followed by non-administration on days 4-14 of the 14-day cycle. In some embodiments, a dosing regimen for intravenous rigosertib can comprise administration on days 1-3 of a 28-day cycle followed by non-administration on days 4-28 of the 28-day cycle. In some embodiments, a dosing regimen for intravenous rigosertib can comprise administration on days 1-3 of a 14-day cycle, followed by non-administration on days 4-14 of the 14-day cycle, then on days 1-3 of a 28-day cycle and non-administration on days 4-28 of the 28-day cycle thereafter, with the 28-day cycle optionally repeating any number of times. In some embodiments, a dosing regimen for intravenous rigosertib can comprise increasing doses on days 1-3 of a 14-day cycle. In some embodiments, a dosing regimen for intravenous rigosertib can comprise increasing doses on days 1-3 of a 28-day cycle.

In some embodiments, a dosing regimen can increase the dose to 2100 mg/day. In some embodiments, a dosing regimen can increase the dose to 2400 mg/day. In some embodiment, a dosing regimen can increase a dose until the MTD and RP2D are established according to 3+3 dose escalation.

A compound disclosed herein can be administered via subcutaneous or intravenous injection. The volume of an injection can be about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, about 2 mL, about 2.1 mL, about 2.2 mL, about 2.3 mL, about 2.4 mL, about 2.5 mL, about 2.6 mL, about 2.7 mL, about 2.8 mL, about 2.9 mL, or about 3 mL. The individual dose administered to a subject can be about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg.

Pharmaceutical compositions described herein can be in unit dosage forms suitable for single administration of precise dosages. In unit dosage form, the formulation can be divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for parenteral injection can be presented in unit dosage form, for example, in ampoules, or in multi-dose containers with a preservative.

A compound described herein can be present in a composition in a range of from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, or from about 250 mg to about 300 mg.

A compound described herein can be present in a composition in an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 225 mg, about 250 mg, or about 300 mg.

A compound described herein can be administered to a subject in an amount of about 0.1 mg/kg to about 500 mg/kg, about 1 mg/kg to about 500 mg/kg, about 0.1 mg/kg to about 300 mg/kg, about 1 mg/kg to about 300 mg/kg, or about 0.1 mg/kg to about 30 mg/kg. In some embodiments, the compound disclosed herein is administered to a subject in an amount of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 120 mg/kg, about 150 mg/kg, about 160 mg/kg, about 180 mg/kg, about 200 mg/kg, about 240 mg/kg, about 250 mg/kg, about 300 mg/kg, about 350 mg/kg, about 360 mg/kg, about 400 mg/kg, about 450 mg/kg, about 500 mg/kg, or about 600 mg/kg of the subject.

A compound described herein can be administered before, during, or after the occurrence of a disease or condition, and the timing of administering the composition containing a compound can vary. For example, a compound can be used as a prophylactic and can be administered continuously to subjects with a propensity to conditions or diseases to lessen or reduce a likelihood of the occurrence of the disease or condition. A compound and composition can be administered to a subject during or as soon as possible after the onset of the symptoms. The administration of a compound can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, or about 25 years. The length of treatment can vary for each subject.

In some embodiments, a dosing regimen can comprise administration for a first period followed by a period of not receiving administration of a compound of the disclosure. In some embodiments, the first period wherein a compound of the disclosure is administered is at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, or at least about 30 days. In some embodiments, the first period wherein a compound of the disclosure is administered is about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days. In some embodiments, the first period wherein a compound of the disclosure is administered is about 1 day. In some embodiments, the first period wherein a compound of the disclosure is administered is about 3 days. In some embodiments, the first period wherein a compound of the disclosure is administered is about 5 days. In some embodiments, the first period wherein a compound of the disclosure is administered is about 7 days. In some embodiments, the first period wherein a compound of the disclosure is administered is about 10 days. In some embodiments, the first period wherein a compound of the disclosure is administered is about 14 days. In some embodiments, the first period wherein a compound of the disclosure is administered is about 21 days. In some embodiments, the first period wherein a compound of the disclosure is administered is about 28 days.

In some embodiments, the second period wherein a compound of the disclosure is not administered (i.e., break between treatment periods) is at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, or at least about 30 days. In some embodiments, the second period wherein a compound of the disclosure is not administered (i.e., break between treatment periods) is about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days. In some embodiments, the second period wherein a compound of the disclosure is not administered (i.e., break between treatment periods) is about 1 day. In some embodiments, the second period wherein a compound of the disclosure is not administered (i.e., break between treatment periods) is about 5 days. In some embodiments, the second period wherein a compound of the disclosure is not administered (i.e., break between treatment periods) is about 7 days. In some embodiments, the second period wherein a compound of the disclosure is not administered (i.e., break between treatment periods) is about 11 days. In some embodiments, the second period wherein a compound of the disclosure is not administered (i.e., break between treatment periods) is about 14 days. In some embodiments, the second period wherein a compound of the disclosure is not administered (i.e., break between treatment periods) is about 21 days. In some embodiments, the second period wherein a compound of the disclosure is not administered (i.e., break between treatment periods) is about 25 days.

In some embodiments, a treatment cycle comprises a first period when a compound of the disclosure is administered and a second period wherein the compound is not administered. In some embodiments, a treatment cycle can be at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, at least about 32 days, at least about 33 days, at least about 34 days, at least about 35 days, at least about 36 days, at least about 37 days, at least about 38 days, at least about 39 days, at least about 40 days, at least about 41 days, at least about 42 days, at least about 43 days, at least about 44 days, at least about 45 days, at least about 46 days, at least about 47 days, at least about 48 days, at least about 49 days, or at least about 50 days. In some embodiments, a treatment cycle can be about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, or about 50 days. In some embodiments, a treatment cycle can be about 14 days, or 2 weeks. In some embodiments, a treatment cycle can be about 21 days, or 3 weeks. In some embodiments, a treatment cycle can be about 28 days, or 4 weeks.

In some embodiments, a dosing regimen cycle can be administered for more than one cycle. In some embodiments, a dosing regimen of the disclosure can be administered for at least 1, at least 2, at least 3, at least 4, at least 5, at least 6, at least 7, at least 8, at least 9, at least 10, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, or more cycles. In some embodiments, a dosing regimen of the disclosure can be administered for 1, 2, 3, 4, 5, 6, 7, 8, 9, 10, 12, 13, 14, 15, 16, 17, 18, 19, 20, 21, 22, 23, 24, 25, 26, 27, 28, 29, 30, or more cycles. In some embodiments, a dosing regimen of the disclosure can be administered for 1 cycle. In some embodiments, a dosing regimen of the disclosure can be administered for 3 cycles. In some embodiments, a dosing regimen of the disclosure can be administered for 5 cycles. In some embodiments, a dosing regimen of the disclosure can be administered for 8 cycles. In some embodiments, a dosing regimen of the disclosure can be administered for 10 cycles. In some embodiments, a dosing regimen of the disclosure can be administered for 12 cycles. In some embodiments, a dosing regimen of the disclosure can be administered for 15 cycles.

In some embodiments, a dosing regimen for a compound of the disclosure can comprise administration on days 1-3 of a 14-day cycle. In some embodiments, a dosing regimen for a compound of the disclosure can comprise administration on days 1-3 of a 28-day cycle. In some embodiments, a dosing regimen for a compound of the disclosure can comprise administration on days 1-3 of a 14-day cycle, then on days 1-3 of a 28-day cycle thereafter. In some embodiments, a dosing regimen for a compound of the disclosure can comprise increasing doses on days 1-3 of a 14 day cycle. In some embodiments, a dosing regimen for a compound of the disclosure can comprise increasing doses on days 1-3 of a 28 day cycle.

In some embodiments, a method of the disclosure can comprise a drug holiday between treatment cycles. In some embodiments, a drug holiday can be at least about 1 week at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 15 weeks, at least about 16 weeks, at least about 17 weeks, at least about 18 weeks, at least about 19 weeks, or at least about 20 weeks. In some embodiments, a drug holiday can be about 1 week about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, or about 20 weeks.

In some embodiments, the administering is once a day. In some embodiments, the administering is twice a day. In some embodiments, the administering is three times a day. In some embodiments, the administering is four times a day. In some embodiments, the administering occurs in the morning. In some embodiments, the administering occurs in the afternoon. In some embodiments, the administering occurs in the evening. In some embodiments, the administering occurs in the morning and in the afternoon. In some embodiments, the administering occurs in the morning and in the evening. In some embodiments, the administering occurs in the afternoon and in the evening.

In some embodiments, the subject is in a fasted state in the morning when a compound of the disclosure is administered to the subject. In some embodiments, the subject is in a fed state in the afternoon when a compound of the disclosure is administered to the subject. In some embodiments, the subject is in a fed state in the evening when a compound of the disclosure is administered to the subject. In some embodiments, the administering occurs in the morning and in the afternoon, wherein the subject is in a fasted state in the morning and in a fed state in the afternoon. In some embodiments, the administering occurs in the morning and in the evening, wherein the subject is in a fasted state in the morning and in a fed state in the evening. In some embodiments, the administering occurs in the afternoon and in the evening, wherein the subject is in a fed state in the afternoon and in a fed state in the evening.

Non-limiting examples of a dosing schedule for administration of a compound described herein include once daily (QD), twice daily (BID), three times daily (TID), four times daily (QID), once weekly, twice weekly, three times weekly, once monthly, twice monthly, and once every other month.

### EXAMPLES

### Example 1: Clinical Study to Evaluate an Oral and Intravenous Pharmaceutical Composition Disclosed Herein for Treating a Disease in a Subject

A phase 2, open-label single arm was conducted. The study was designed to assess the efficacy and safety of Rigosertib in patients with recessive dystrophic epidermolysis bullosa (RDEB)-associated locally advanced/metastatic squamous cell carcinoma (SCC).

In addition to the assessment of safety and tolerability, efficacy was measured by the overall response rate as assessed by RECIST. Patients were treated until RECIST progression criteria were met (i.e. 20% increase in tumor diameter or appearance of new malignant lesions) for 12 consecutive months or until an unacceptable toxicity or intolerance, or until remission.

Study Design: Rigosertib was administered in two different formulations: oral capsules, and IV solution for intravenous infusion. Rigosertib was provided as 280 mg capsules and as a liquid solution.

The study had two primary endpoints. The first was to determine the anti-tumor activity of rigosertib in RDEB patients with advanced SCC who had failed prior standard of care through the overall response rate (ORR), defined as the proportion of patients who achieved either a complete response (CR) or a partial response (PR). The second primary endpoint was to evaluate the safety and tolerability of rigosertib in the population. Secondary study endpoints included quality of life and a biomarker analysis performed on archival tissue from all patients. Patients were dosed for up to one year, with trial duration anticipated to be approximately two-and-a-half years.

Oral dosing: Study subjects were administered oral Rigosertib continuously for a total of 3 weeks, every four week cycle (three weeks on, one week off drug). Study subjects received 560 mg of oral Rigosertib (i.e. 2 capsules of 280 mg) in the morning and 560 mg of oral Rigosertib (i.e. 2 capsules of 280 mg) in the afternoon, total of 1120 mg/day.

Intravenous dosing: For continuous IV infusion as Rigosertib, sodium salt concentrate, 75 mg/mL, was formulated to contain 75 mg of Rigosertib sodium salt per mL (equivalent to 75 mg of Rigosertib sodium per mL) as a sterile solution in polyethylene glycol 400 (PEG 400) containing a small amount of sodium hydroxide for pH adjustment. Rigosertib.Na 75 mg/mL was packaged in single-use glass vials with Teflon-lined rubber stoppers containing 24 mL of drug product (1800 mg). Just prior to dosing, the Rigosertib concentrate (i.e. 1 glass vial, 24 mL) was diluted to a final volume of 500 ml in 0.9% sodium chloride for injection.

Efficacy Assessments: Overall response rate (ORR) was defined as the proportion of patients who achieved either a CR or a PR. Responses of complete remission (CR), partial remission (PR), mCR, SD, failure, and PD were determined by RECIST criteria. The number and percent of patients with CR, PR, mCR, SD, Failure or PD were summarized.

Response per RECIST criteria: Efficacy assessments included an initial tumor assessment at week 12 (±3 days) after first dose. Further tumor assessments were completed every 12 weeks (±3 days). Study evaluations were performed according to RECIST 1.1 criteria. High resolution CT with oral or IV contrast or contrast-enhanced MRI were used as imaging modalities for assessing radiographic tumor response. Screening/Baseline assessments were within 4 weeks of first dose of study drug. Brain MRI was used as an imaging method for evaluating CNS metastasis, and an assessment was required during screening/baseline and End of Therapy or as clinically indicated.

Target lesions: When more than one measurable lesion was present at baseline, all lesions up to a maximum of 5 lesions total (and a maximum of two lesions per site), representative of all involved sites, were investigated as target lesions and are recorded and measured at baseline. Target lesions were selected on the basis of size (lesions with the longest diameter) and were representative of all involved sites. A sum of the diameters (longest for non-nodal lesions, short axis for nodal lesions) for all target lesions were calculated and reported as the baseline sum diameters. If lymph nodes were included in the sum, only the short axis was added into the sum. The baseline sum diameters were used as reference to characterize any objective tumor regression in the measurable dimension of the disease.

Quality of Life: Quality of life was measured by the QOLEB questionnaire, which was administered at baseline and every week thereafter. Baseline and change from baseline results were summarized by visit using descriptive statistics for this overall measure.

Results: A clinical exam and imaging studies showed the complete resolution of two of three squamous cell target lesions and a reduction in the third target lesion following treatment of a REDB patient with intravenous rigosertib. Two target lesions on the left hand disappeared completely visually and by MRI. A third target lesion on the right elbow apparently diminished but did not disappeared. These data were obtained from a 12 week MRI and visit at the clinic.

### Example 2: Rigosertib for Locally Advanced Metastatic EB-Associated SCC

RDEB is a severe genodermatosis caused by mutations in COL7A1 and characterized by generalized skin blistering and involvement of mucous membranes. Aggressive, metastasizing squamous cell carcinomas (SCCs) typically arising in areas of chronic skin wounding and inflammation are the primary cause of death in this cohort. These SCCs show limited response rates of mostly short duration to conventional chemotherapy and radiotherapy and targeted therapy with anti-epidermal growth factor antibody (cetuximab, panitumumab) and tyrosine kinase inhibitors (gefitinib, erlotinib). Preclinical models (1) demonstrated that RDEB SCC keratinocytes are specifically sensitive to PLK 1 reduction by siRNA treatment compared to normal primary RDEB keratinocytes and (2) further identified the PI3K/PLK1 inhibitor Rigosertib as having the best specificity by demonstrating the largest therapeutic window separating tumor and normal cells.

Eligible patients showed histologically confirmed, locally advanced/metastatic SCCs that have failed prior standard of care, including immune checkpoint inhibitors. The treatment regimen was 72 hr CIV infusions of initially 1800 mg/24 h on days 1-3 of a two week cycle for 8 cycles, and then on days 1-3 of a 4 week cycle for 9 cycles. Patients were treated as described above in EXAMPLE 1.

The patient was a 24 year old RDEB-patient with multiple, histologically-confirmed, unresectable, cutaneous SCCs. Disease continued to progress under prior treatment with cemiplimab. Rigosertib application led to sustained clinical and histological remission of all target lesions without signs of metastatic disease as assessed after trial completion (17 treatment cycles).

Safety and tolerability of rigosertib were favorable. Observed adverse effects included CTCAE grade II irritative cystitis, alopecia, recurrent episode of bacteriaemia with predominantly S. aureus and P. aeruginosa (most probably due to chronic skin wounding and indwelling catheters for CIV rigosertib infusions, however without cardiovascular-systemic compromise), nausea, increased lesional pain, and amenorrhea. All the side effects were expected based on safety data from other, non-EB trials and were characteristic for cytotoxic treatment approaches. The initial, single patient indicated anti-tumor activity and acceptable safety profile of rigosertib in the setting of EB-associated SCC with usually fatal prognosis.

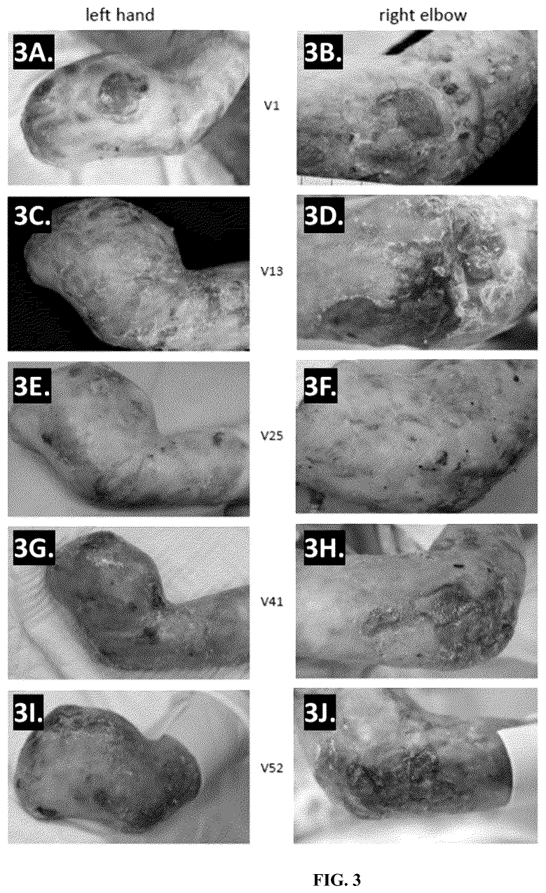

FIG. 3 shows clinical photography images of target legions on the left hand (Panels 3A, 3C, 3E, 3G, and 3I) and the right elbow (Panels 3B, 3D, 3F, 3H, and 3J) at V1 (day 1), V13 (days 85-95), V25 (day 169-175), V41 (days 281-283), and V52 (d358-364). At V25, both lesions were biopsied with no histological signs of malignancy. On V41, a wound was noticed on the right elbow. The wound showed an EB-typical course with complete healing within several weeks.

### EMBODIMENTS

The following non-limiting embodiments provide illustrative examples of the invention, but do not limit the scope of the invention.

Embodiment 1. A method of treating a condition in a subject in need thereof, the method comprising administering to the subject a therapeutically-effective amount of a compound of the formula:

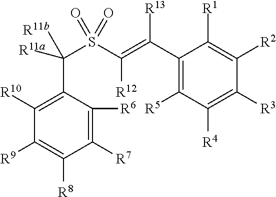

or a pharmaceutically-acceptable salt or zwitterion thereof,
wherein:
   a. each R¹, R², R³, R⁴, R⁵, R⁶, R⁷, R⁸, R⁹, R¹⁰, R^{11a}, R^{1b}, R¹², and R¹³ is independently alkyl, alkenyl, alkynyl, alkoxy, aryl, heteroaryl, heterocyclyl, C(O)R^{x}, C(O)OR^{x}, C(O)NR^{x}R^{y}, OR^{x}, SR^{x}, NR^{x}R^{y}, NR^{x}C(O)R^{Y}, OC(O)R^{x}, or SiR^{x}R^{y}R^{z}, each of which is independently substituted or unsubstituted; or hydrogen or halogen; and
   b. each R^{x}, R^{y}, and R^{z} is independently alkyl, alkenyl, alkynyl, aryl, heteroaryl, or heterocyclyl, each of which is independently substituted or unsubstituted; or hydrogen or halogen,
   c. wherein the condition is recessive dystrophic epidermolysis bullosa, and
   d. wherein within about 12 weeks of the administering, a lesion in an area of skin associated with the recessive dystrophic epidermolysis bullosa resolves as determined by magnetic resonance imaging of the area of skin.

Embodiment 2. The method of embodiment 1, wherein within about 8 weeks of the administration, the lesion in the area of skin associated with recessive dystrophic epidermolysis bullosa resolves as determined by magnetic resonance imaging of the area of skin.

Embodiment 3. The method of embodiment 1, wherein within about 4 weeks of treatment, the lesion in the area of skin associated with recessive dystrophic epidermolysis bullosa resolves as determined by magnetic resonance imaging of the area of skin.

Embodiment 4. The method of embodiment 1, wherein within about 2 weeks of treatment, the lesion in the area of skin associated with recessive dystrophic epidermolysis bullosa resolves as determined by magnetic resonance imaging of the area of skin.

Embodiment 5. The method of any one of embodiments 1-4, wherein the recessive dystrophic epidermolysis bullosa is associated with a squamous cell carcinoma.

Embodiment 6. The method of any one of embodiments 1-5, wherein the lesion is squamous cell carcinoma.

Embodiment 7. The method of any one of embodiments 1-6, wherein the subject is refractory against a standard of care therapy for recessive dystrophic epidermolysis bullosa.

Embodiment 8. The method of any one of embodiments 1-7, wherein the subject is refractory against a standard of care therapy for a squamous cell carcinoma.

Embodiment 9. The method of any one of embodiments 1-8, wherein the administering is oral.

Embodiment 10. The method of any one of embodiments 1-9, wherein the compound is formulated as a capsule.

Embodiment 11. The method of any one of embodiments 1-10, wherein the compound is formulated as a soft gel capsule.

Embodiment 12. The method of any one of embodiments 1-8, wherein the administering is intravenous.

Embodiment 13. The method of embodiment 1 or 12, wherein the administering is a 72 hour intravenous infusion.

Embodiment 14. The method of any one of embodiments 1-8, wherein the administering is intratumoral.

Embodiment 15. The method of any one of embodiments 1-14, wherein the administering is on each of days 1, 2, and 3 of a two-week cycle.

Embodiment 16. The method of any one of embodiments 1-14, wherein the administering is on each of days 1, 2, and 3 of a two-week cycle for 8 cycles, then on each of days 1, 2, and 3 of a 4-week cycle.

Embodiment 17. The method of any one of embodiments 1-14, wherein the administering is on a four-week cycle of daily administration for three weeks followed by one week with no administrations.

Embodiment 18. The method of any one of embodiments 1-17, wherein each R¹, R³, and R⁵ is independently OR^{x}.

Embodiment 19. The method of embodiment 18, wherein each R^{x} is independently alkyl, aryl, heteroaryl, or heterocyclyl, each of which is independently substituted or unsubstituted.

Embodiment 20. The method of embodiment 18, wherein each R^{x} is independently substituted or unsubstituted C₁₋₆ alkyl.

Embodiment 21. The method of embodiment 20, wherein each Rx is independently C₁ alkyl substituted with hydroxy, sulfhydryl, halogen, an amino group, a nitro group, cyano, a sulfoxide group, a sulfone group, a sulfonamide group, a carboxyl group, a carboxylic acid, a carboxaldehyde group, alkoxy, aryl, a heterocyclyl group, an acyl group, an amide, or an ester.

Embodiment 22. The method of embodiment 18, wherein each R^{x} is independently methyl.

Embodiment 23. The method of any one of embodiments 1-22, wherein each R², R⁴, R⁶, R⁹, and R¹⁰ is independently hydrogen.

Embodiment 24. The method of any one of embodiments 1-23, wherein each R^{11a} and R^{11b} is independently hydrogen.

Embodiment 25. The method of any one of embodiments 1-24, wherein each R¹² and R¹³ is independently hydrogen.

Embodiment 26. The method of any one of embodiments 1-25, wherein the compound has the formula:

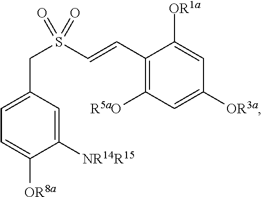

wherein:
each R^{1a}, R^{3a}, R^{5a}, and R^{8a} is independently substituted or unsubstituted C₁₋₈ alkyl; and
each R¹⁴ and R¹⁵ is independently alkyl, alkoxy, aryl, heteroaryl, or heterocyclyl, each of which is independently substituted or unsubstituted; or hydrogen.

Embodiment 27. The method of embodiment 26, wherein each R^{1a}, R^{3a}, R^{5a}, and R^{8a} is independently methyl.

Embodiment 28. The method of embodiment 26, wherein R¹⁴ is hydrogen.

Embodiment 29. The method of embodiment 26, wherein R¹⁵ is C₁ alkyl substituted with hydroxy, sulfhydryl, halogen, an amino group, a nitro group, cyano, a sulfoxide group, a sulfone group, a sulfonamide group, a carboxyl group, a carboxylic acid, a carboxaldehyde group, alkoxy, aryl, a heterocyclyl group, an acyl group, an amide, or an ester.

Embodiment 30. The method of embodiment 26, wherein R¹⁵ is CH₂COOH.

Embodiment 31. The method of any one of embodiments 1-17, wherein the compound is (E)-2-(5-((2,4,6-trimethoxystyrylsulfonyl)methyl)-2-methoxyphenylamino)acetic acid, or a pharmaceutically-acceptable salt or zwitterion thereof.

Embodiment 32. The method of any one of embodiments 1-17, wherein the compound is sodium (E)-2-(5-((2,4,6-trimethoxystyrylsulfonyl)methyl)-2-methoxyphenylamino)acetate.

Embodiment 33. The method of any one of embodiments 1-32, wherein the therapeutically-effective amount is from about 280 mg to about 2400 mg.

Embodiment 34. The method of any one of embodiments 1-32, wherein the therapeutically-effective amount is about 280 mg.

Embodiment 35. The method of any one of embodiments 1-32, wherein the therapeutically-effective amount is about 560 mg.

Embodiment 36. The method of any one of embodiments 1-32, wherein the therapeutically-effective amount is about 840 mg.

Embodiment 37. The method of any one of embodiments 1-32, wherein the therapeutically-effective amount is about 1120 mg.

Embodiment 38. The method of any one of embodiments 1-32, wherein the therapeutically-effective amount is about 1800 mg.

Embodiment 39. The method of any one of embodiments 1-32, wherein the therapeutically-effective amount is about 2100 mg.

Embodiment 40. The method of any one of embodiments 1-32, wherein the therapeutically-effective amount is about 2400 mg.

Embodiment 41. The method of any one of embodiments 1-40, wherein the administering is once a day.

Embodiment 42. The method of any one of embodiments 1-40, wherein the administering is twice a day.

Embodiment 43. The method of any one of embodiments 1-40, wherein the administering is three times a day.

Embodiment 44. The method of any one of embodiments 1-40, wherein the administering is oral on a four-week cycle of: i) three weeks of about 560 mg per morning and 560 mg per evening; ii) one week of no administration.

Embodiment 45. The method of any one of embodiments 1-40, wherein the administering occurs at a morning time and at an evening time, wherein the subject is in a fasted state at the morning time and the subject is in a fed state at the evening time.

Embodiment 46. The method of any one of embodiments 1-8, 12-45, wherein the therapeutically-effective amount is about 1,800 mg, and the compound is diluted in 0.9% saline.

Embodiment 47. The method of any one of embodiments 1-46, further comprising assaying the subject for a biomarker of a PI3K pathway prior to the administering.

Embodiment 48. The method of any one of embodiments 1-47, further comprising assaying the subject for a biomarker of an Akt pathway prior to the administering.

Embodiment 49. The method of any one of embodiments 1-48, further comprising assaying the subject for a biomarker of a PLK1 pathway prior to the administering.

Embodiment 50. The method of any one of embodiments 1-49, wherein the subject has an esophageal obstruction.

1-50. (canceled)

51. A method of treating a condition in a subject in need thereof, the method comprising administering to the subject a therapeutically-effective amount of (E)-2-(5-((2,4,6-trimethoxystyrylsulfonyl)methyl)-2-methoxyphenylamino)acetic acid or a pharmaceutically-acceptable salt or zwitterion thereof,
wherein the condition is recessive dystrophic epidermolysis bullosa, and
wherein within about 12 weeks of the administering, a lesion in an area of skin associated with the recessive dystrophic epidermolysis bullosa resolves as determined by assay.

52. The method of claim 51, wherein within about 4 weeks of treatment, the lesion in the area of skin associated with recessive dystrophic epidermolysis bullosa resolves as determined by magnetic resonance imaging of the area of skin.

53. The method of claim 51, wherein the recessive dystrophic epidermolysis bullosa is associated with a squamous cell carcinoma.

54. The method of claim 51, wherein the lesion is squamous cell carcinoma.

55. The method of claim 51, wherein the subject is refractory against a standard of care therapy for recessive dystrophic epidermolysis bullosa.

56. The method of claim 51, wherein the subject is refractory against a standard of care therapy for a squamous cell carcinoma.

57. The method of claim 51, wherein the administering is oral.

58. The method of claim 51, wherein the administering is intravenous.

59. The method of claim 51, wherein the administering is a 72 hour intravenous infusion.

60. The method of claim 51, wherein the administering is intratumoral.

61. The method of claim 51, wherein the administering is on each of days 1, 2, and 3 of a two-week cycle.

62. The method of claim 51, wherein the administering is on each of days 1, 2, and 3 of a two-week cycle for 8 cycles, then on each of days 1, 2, and 3 of a 4-week cycle.

63. The method of claim 51, wherein the administering is on a four-week cycle of daily administration for three weeks followed by one week with no administrations.

64. The method of claim 51, wherein the compound is sodium (E)-2-(5-((2,4,6-trimethoxystyrylsulfonyl)methyl)-2-methoxyphenylamino)acetate.

65. The method of claim 51, wherein the therapeutically-effective amount is from about 280 mg to about 2400 mg.

66. The method of claim 51, wherein the administering is oral on a four-week cycle of: i) three weeks of about 560 mg per morning and 560 mg per evening; ii) one week of no administration.

67. The method of claim 51, wherein the administering occurs at a morning time and at an evening time, wherein the subject is in a fasted state at the morning time and the subject is in a fed state at the evening time.

68. The method of claim 51, further comprising assaying the subject for a biomarker of a PI3K pathway prior to the administering.

69. The method of claim 51, further comprising assaying the subject for a biomarker of an Akt pathway prior to the administering.

70. The method of claim 51, further comprising assaying the subject for a biomarker of a PLK1 pathway prior to the administering.

71. The method of claim 51, further comprising assaying the subject for a biomarker of a COL7A1 pathway prior to administering.

72. The method of claim 51, wherein the assay comprises magnetic resonance imaging of the area of skin.
